(19) United States
(12) Patent Application Publication
(10) Pub. No.: US 2014/0066804 A1
(43) Pub. Date: Mar. 6, 2014

(54) BLADDER PRESSURE MEASUREMENT SYSTEM

(71) Applicants: Sebastian Wille, Koeln (DE); Dirk Tenholte, Chemnitz (DE); Jan Mehner, Neukirchen (DE)

(72) Inventors: Sebastian Wille, Koeln (DE); Dirk Tenholte, Chemnitz (DE); Jan Mehner, Neukirchen (DE)

(21) Appl. No.: 14/002,136

(22) PCT Filed: Mar. 14, 2012
(86) PCT No.: PCT/EP2012/001125

§ 371 Date: Oct. 22, 2013

(30) Foreign Application Priority Data
Mar. 17, 2011 (DE) 102011014220.7

(51) Int. Cl. A61B 5/00; A61B 5/20

(52) U.S. Cl. CPC A61B 5/6874; A61B 5/205
USPC 600/561

(57) ABSTRACT

The invention relates to a bladder pressure measurement system and to a measurement method comprising a measurement capsule (1) that can be placed in the bladder of a living creature, in particular of a human being, and comprising a pressure sensor and measurement electronics, by means of which pressure measurement values provided by the pressure sensor can be captured and saved, comprising a probe device having a manually actuated probe, wherein the probe device and the measurement capsule comprise internal chronometers synchronized to each other or at least capable of being synchronized to each other and the measurement capsule (1) is designed to capture and save pressure measurement values together with time information from the chronometer thereof, and the probe device is designed to detect and save a probe actuation together with time information from the chronometer thereof.

The invention relates to a bladder-pressure measurement system comprising a measurement capsule that can be placed inside the bladder of a living creature, in particular of a human being, and including a pressure sensor and measurement electronics by means of which pressure measurement values provided by the pressure sensor can be captured and stored.

The invention further relates to a method of measuring the pressure inside the bladder, by means of a measurement capsule placed inside the bladder of a living creature, in particular a human being, and including a pressure sensor and measurement electronics that can capture and store pressure measurement values provided by the pressure sensor.

The prior art teaches that many patients present with clinical symptoms suggesting a compromised bladder storage or bladder emptying function. However, standard urodynamic testing often does not yield any pathological findings.

This is often associated with the fact that the usual standardized urodynamic tests, involving a urethral and a rectal measuring catheter, are only performed for the duration of a narrow time frame that lasts only 20 to 40 minutes, during which changes of the vesical and abdominal pressure values are captured as a function of the bladder filling. Due to the shortness of this window over time, the probability of capturing pathological pressure changes with sufficient certainty is comparatively small.

For the implementation of long-term urodynamic testing, the prior art further teaches admitting patients to the hospital and conducting the above-described same measurement method, just over a longer period of time. However, in many instances, this also does not yield meaningful results, because such long-term urodynamic testing requires that patients be removed from their normal routine and surroundings, or a situation that would resemble their reality, which is why they do not react in the way they would normally react.

Another method of conducting long-term urodynamic testing uses a measurement capsule that is introduced into a living creature to capture and store measured values of the prevailing pressure inside the bladder over the course of a longer period of time.

Although bladder-pressure measurement systems that comprise such a measurement capsule and/or such a measurement method that utilizes such a measurement capsule have the advantage that they allow for conducting long-term urodynamic testing in the patient's normal surroundings and in routine everyday situations, problems result, however, from the placement of the measurement capsule itself.

A bladder-measurement system and method of the above-described type is known in the art, for example, from the publication “An Autonomous, Capacitive Sensor Based and Battery Powered Internal Bladder Pressure Monitoring System” by Philippe Jourand and Robert Puers in ScienceDirect, Procedia Chemistry 1 (2009) 1263-1266.

The authors, Jourand and Puers, describe a measurement system that comprises a measurement capsule that is placed inside the bladder in a known manner; however, the publication fails to address entirely the problems associated with such a measurement capsule that can be placed nonsurgically inside the bladder, for example, by a cystoscope or an application aid. These risks include that such a measurement capsule can be expelled into the urethra during miction, it may clog the urethra, may be lost as a measurement capsule, or that it comes to lie in front of the bladder outlet, thereby blocking the outflow of urine.

U.S. publication 2010/0121161 discloses another known system from the prior art where a measurement capsule is used that can be placed inside the bladder of a human being and where the emplaced measurement capsule includes a sleeve that is inflated through a catheter from the outside, or that is inflated, employing an alternate solution, by gas from two components that are carried along inside the measurement capsule and chemically react to release gas.

While an inflated measurement capsule as provided herein is able to prevent expulsion of the capsule into the urethra, the problem that the inflated measurement capsule will block the bladder outlet to the urethra, if the inflated capsule places itself against the outlet, still persists.

Moreover, inflating the measurement capsule is complicated and only possible by employing additional measures from the outside or by, if necessary, auxiliary means that are carried along inside the measurement capsule that are not always harmless.

Furthermore, this publication does not describe a satisfactory option for removing the measurement capsule from the bladder. All that is mentioned is that the inflated sleeve of the measurement capsule includes a suture that will dissolve over time, such that the gas is able to escape from the sleeve, the sleeve of the measurement capsule then collapses and is then automatically expelled when the bladder is voided during miction.

The problem that arises, correspondingly, is the fact that the exact point in time when the sleeve of the measurement capsule will collapse cannot be predicted, which is why there is also the risk that the measurement capsule is lost during miction.

Therefore, it is the object of the present invention to provide a method of the kind as described above that will preclude the above-described risks associated with a measurement capsule according to the prior art, where such a measurement capsule cannot inadvertently enter the urethra or block the bladder outlet, and where, moreover, controlled recovery of the measurement capsule is possible.

It is also the object of the present invention to provide a bladder-pressure measurement system of the kind known from the prior art that is more reliable in terms of capturing the measured values.

According to one aspect of the invention, this object is achieved in that a measurement capsule of the previously described class is provided with at least one locking element that has a force applied thereto; particularly, the locking element is disposed in a loss-proof manner and can be placed, acting against the effective force, against the measurement capsule, and it can be automatically pushed away from the measurement capsule by the effective force, at least locally, in particular whereby the external cross-section of the measurement capsule can be, at least locally, enlarged.

A loss-proof apparatus can be achieved, for example, by providing that a locking element is movably fastened to the measurement capsule, for example in an articulated manner.

Correspondingly, when the at least one locking element rests against the measurement capsule, the measurement capsule of the system according to the invention has an outside diameter that allows for passing the measurement capsule through the urethra and into the bladder, as well as removing it from the bladder. In this state, when the at least one locking element is pushed away, at least locally, from the measurement capsule by the effective force, the outside diameter is, at least locally, enlarged such that the measurement capsule cannot be lost through the urethra or block the bladder outlet.

It can be provided therein, for example, that during the nonsurgical placement or removal of the measurement capsule through the urethra, the urethra itself is instrumental in pressing the at least one locking element against the measurement capsule, counteracting the effective force.

In one possible embodied example, it can also be provided that the at least one locking element can be pressed against the measurement capsule by a sleeve that surrounds and/or can be fitted around the measurement capsule, and that can be pushed away automatically from the measurement capsule after removal of the sleeve.

Accordingly, a measurement capsule of this kind according to the invention can be implemented such that, when it is in the position it assumes when in the above-described sleeve, it can be placed inside the bladder in the usual manner, particularly without surgical intervention, through the urethra and into the bladder, for example by a cystoscope or an application aid.

Preferably, the measurement capsule can be dimensioned such that the external cross-section thereof is smaller than the free internal cross-section of the used cystoscope or application aid (for example, a catheter), such that the measurement capsule can be pushed by the cystoscope or the application aid through the urethra and into the bladder. Advantageously, a front, hollow end region of the cystoscope receives the measurement capsule, and this hollow end region constitutes the above-described sleeve.

This has the advantage that, for as long as the measurement capsule is located in the front, hollow end region of the cystoscope or application aid, the hollow end region functions effectively as a sleeve and ensures that the at least one locking element is pressed against the measurement capsule, counteracting the application of force applied thereto.

Therefore, with the placed, at least one locking element, the measurement capsule can be expelled from the hollow end region of the cystoscope or application aid and it thereby reaches the interior of the bladder. The sleeve that previously formed the hollow end of the cystoscope or catheter is removed by this expelling action, such that, due to the application of force, the at least one locking element is removed automatically from the surface of the measurement capsule, meaning it is pushed away by the force that preferably acts from the measurement capsule toward the outside, such that the external cross-section of the measurement capsule is, at least locally, enlarged, namely where the at least one locking element is provided.

The essential advantage of the measurement capsule of the claimed bladder-pressure measurement system according to the invention in contrast to the prior art lies in the fact that, by enlarging the outside diameter, the measurement capsule according to the invention cannot be expelled into the urethra.

Moreover, since the at least one locking element does not have a no closed surface, as described in the context of the inflatable sleeve with regard to the measurement capsule as referred to in the introduction, a measurement capsule of this kind according to the invention would be unable to block the bladder outlet, even if the measurement capsule inadvertently slips in front of the bladder outlet, because urine will still be able to find a path to the bladder outlet through the space between the at least one locking element and the surface of the measurement capsule.

Aside from the previously described embodiment, where the hollow end region of a cystoscope or of another application aid constitutes a part of the bladder-pressure measuring system according to the invention, namely in that the end region constitutes the previously described sleeve, it is possible to provide a measurement capsule of the kind according to the invention with a separate, removable sleeve that surrounds the measurement capsule and the at least one locking element, and that can be removed again, particularly in a preferred embodiment, after placement of the measurement capsule inside the bladder.

A removal of this kind can be achieved, for example, by an auxiliary instrument that is introduced into the bladder through the cystoscope or an application aid. Here too, it is provided that, by removing the sleeve, the at least one locking element is pushed away automatically from the measurement capsule by the force that is in effect so the external cross-sectional size is at least partially enlarged.

Such an embodiment of the measurement capsule with a separate, removable sleeve can be advantageous, for example, when the external cross-section and/or the outside diameter of the measurement capsule with the at least one locking element is larger than the free internal cross-section of available cystoscopes, such that in such a case the hollow end thereof cannot receive a measurement capsule. In this case, the separate sleeve holds the at least one locking element in an adjacent resting position against the measurement capsule; and the enveloped measurement capsule therein can be held, for example, by an auxiliary element through a cystoscope and in a position before the hollow end thereof, whereby it is advanced through the urethra into the bladder, and the sleeve is removed only there.

It is considered especially advantageous, when the embodiment of the measurement capsule is configured such that a plurality of locking elements, particularly a plurality of similar or identical locking elements, is distributed at least in one angular direction around the measurement capsule.

It can be envisioned, for example, that a measurement capsule has a longitudinal extension, and in that it is essentially cylindrical in terms of this longitudinal extension, for example, having of circular cross-section perpendicularly relative to its longitudinal extension. Correspondingly, the plurality of locking elements has an even angular distribution around the circular cross-section. It is advantageous for at least three similar or identical locking elements to be provided on the measurement capsule according to the invention.

In one possible embodiment, the locking elements are configured as spring wires that each extend along the longitudinal is extension of a measurement capsule of the previously described longitudinally extended type from the one end to the other end thereof, and where, due to the effect of the spring force present in the spring wire, it is provided at a distance in the center region of the measurement capsule. This can be achieved, for example, by providing that the spacing between the locations on the measurement capsule where the ends of the spring wire are fastened is smaller than the length of the spring wire being used.

The term spring wire is not intended to necessarily indicate that the spring wire is made of metal, although this is possible. A spring wire of this kind can also be made of plastic or a compound material.

Moreover, the term spring wire is not intended to necessarily indicate that the spring wire has a circular cross-section perpendicularly relative to the extension thereof, although this type of cross-section is possible. Rectangular cross-sections are possible as well; for example, also in a configuration where the spring wire has a greater width than height perpendicularly to its longitudinal extension.

A measurement capsule according to the invention of this kind constitutes, after the automatic placement/spacing of the spring wires from the surface of the measurement capsule, a shaped body, when the retaining sleeve has been removed, of a substantially ellipsoid-like shape or ovoidal shape with an outside diameter that is large enough to prevent any such measurement capsule being expelled through the urethra. Moreover, the is measurement capsule only comes into local contact with the inside bladder wall such that voiding urine is still possible, even if the measurement capsule is located in front of the bladder outlet.

To avoid irritation of the bladder wall, for example, the previously mentioned spring wires are coated, particularly with a coating that is well tolerated by mucous membranes. A coating of this kind can be, for example, of silicone. In the alternative, the material can overall be selected as a material that is correspondingly well tolerated, for example stainless steel or titanium.

In another configuration that can also be combined with the previous embodiment, the locking elements can be a two-dimensional element connected to the measurement capsule in an articulated manner, at least on one end. For example, such a two-dimensional element can be carried by one of the preceding respective spring wires, in particular supported, or by any other spring element that generates force that supports itself between the surface of the measurement capsule and the lower side of the two-dimensional element.

A two-dimensional element of this kind can be set at the surface thereof that is directed toward the measurement capsule, as corresponding to the outer contour of the measurement capsule, such that a two-dimensional element of this kind is able to rest closely against the measurement capsule, when it is inside the sleeve. If the measurement capsule is, on the other hand, removed from the sleeve, the two-dimensional element raises itself, powered by the force that is in effect, particularly in the manner of a sail is relative to the surface of the measurement surface.

In one embodiment, in which a two-dimensional element is supported by the above-described spring wire, a plurality of individual two-dimensional elements is provided between the two ends in the direction of extension of the spring wire. This results in a configuration where a segmented belt extends through the locking element between the ends of the measurement capsule. In this context, it can also be provided that the individual two-dimensional elements are displaceably provided on the spring wire. Also, the size of the area of the two-dimensional elements can decrease from the center to an end of the measurement capsule.

The use of such two-dimensional elements has the special advantage that, if a measurement capsule of this kind according to the invention makes contact with the bladder wall, the contact is over larger, two-dimensional regions, such that irritations can be avoided.

Independently of the type and manner of the configuration of the locking elements, particularly if they are configured as described in the context of previous embodiments, it can be provided further that the locking elements are covered, at least locally, by an, in particular, elastic sleeve.

A sleeve of this kind can be embodied, for example, by a silicone sleeve or another type of well tolerated material.

This way, any contact with the bladder wall occurs, if at all, with larger two-dimensional regions, namely formed by the sleeve, thereby avoiding irritation.

In contrast to the measurement capsule that is known from the prior art, which includes an inflatable sleeve, the embodiments of the present kind have, as described, the further advantage that, while the plurality of locking elements supports the sleeve from the inside, enlarging the outside diameter of the measurement capsule according to the invention, however, does not necessarily result in a rotationally symmetrical sleeve, particularly when it is only supported by few locking elements in evenly divided angles from the inside, such that, with this embodiment as well, any blockage of the bladder outlet can be avoided even when the measurement capsule is located in a disadvantageous position.

Furthermore, the embodiment has the advantage that the sleeve of the kind according to the invention is not enlarged via an internal application of pressure by a fluid, but instead by an internal application of force, particularly a spring-type force by the supporting locking elements. Therefore, no additional inflation process is needed, nor is it necessary to carry along any reagents for raising the sleeve.

An elastic sleeve of this kind can also be covered and/or come to be covered by the previously described removable sleeve, at least temporarily during the placement or removal process of the measurement capsule.

In all of the above-described embodiments, it can be provided, in addition, that a gripping aid is provided at least on one of the ends of the longitudinally extended measurement capsule, by means of which the measurement capsule can be pulled into the sleeve, by a gripping element as mentioned in the introduction, pressing in at least one, preferably a plurality of the locking elements.

In particular, when the above-described sleeve is the hollow end of a cystoscope or other application aid, there exists, therefore, the possibility of advancing a gripping element through the cystoscope or application aid into the bladder to capture the gripping aid of the measurement capsule by the gripping element and then retracting the measurement capsule into the sleeve. This way, the previously placed locking elements automatically come to rest again against the measurement capsule and against the effective force, such that, during the retraction step into the hollow end, they automatically reduce their external cross-sectional size and the cystoscope or application aid can be removed from the bladder.

For more ease of application, for example, the hollow end of a cystoscope or an application aid can be provided with a starting bevel that facilitates the placement of the at least one locking element on the outer surface of the measurement capsule.

According to a further aspect of the invention, the reliability of the bladder-pressure measurement system of the invention of a type according to the class, as described in the introduction, as well as, preferably, also of the previously described embodiments is further improved, in that it comprises a probe device with a manually actuated probe, the probe device and the measurement capsule including internal chronometers that can be synchronized with each other, the measurement capsule being set up to capture and store measured pressure values together with time data from the chronometer thereof; and the probe device is set up is such that it captures and stores any probe actuation together with time data from the chronometer thereof. Thereby, it is also possible to store the fact that an actuation of the probe has occurred in that, for example, the time of the actuation is entered in a list, for example only the time or, for example, the length of time that the connection lasted, with a running number or other information.

The essential advantage of such a bladder-pressure measurement system according to the invention must be seen in that, following the placement of the measurement capsule according to the invention with an internal chronometer, in addition, the patient can be handed a probe device, and the patient is asked to actuate the probe of the probe device when the patient experiences urinary urgency. The probe device thus constitutes an additional device of the bladder-pressure measurement system that is separate from the measurement capsule. Due to the fact that these moments of urinary urgency are recorded by the probe device, and the chronometer of the probe device is synchronized with the chronometer of the measurement electronics of the measurement capsule, or it can be synchronized therewith at least, it is possible to arrive at a time comparison of the recorded measured values from the probe device and from the measurement capsule. For example, this way, it is possible to ascertain if the urinary urgency was accompanied by detrusor instability of the bladder, or not.

Only by synchronizing the chronometers of the probe device and of the measurement capsule is it possible to arrive at a direct comparison between the sensation of urinary urgency and the is measured pressure values that were captured during the same time period by the measurement capsule. This way, there is a higher probability that measured values will support the diagnosing physician in his diagnosis.

It can be envisioned that the stored actuations from the probe device, meaning the times when the device was actuated, are read like the measured pressure values from the measurement capsule, as well as the respective times when they were captured, for example after the removal of the capsule from the bladder, or via a wireless connection to a reader device, for example a PC. The actuation times and the measured pressure values can be matched based on the time values.

The reliability of a bladder-pressure measurement system of this kind can be even further improved in that the system further comprises a preurethral placement device that can be positioned in front of the outlet of the urethra of the patient, comprising a sensor for detecting any loss of urine, the preurethral placement device further including an internal chronometer that can be synchronized and/or is synchronized with at least one of the above-described chronometers, the preurethral placement device being set up such that a loss of urine is captured together with time data, as provided by the chronometer thereof.

The fact that a loss of urine has occurred can be stored by, for example, entering this information into a list to record when the loss of urine occurred, for example, only the time or the length of time of connection with a running number or other kind of information.

Correspondingly, a bladder-pressure measurement system according to the invention captures three different types of measured values: these are, namely, the bladder pressure, the actuation of the one or more probes, which is the subjectively experienced urinary urgency, and the actual loss of urine. Since all measured values are provided with a time data/time stamp entry, it is possible to compare these measured values such that diagnosis by a physician can be supported. As described previously, the correlation can be achieved in that the times and, possibly, the measured values are read from the three individual instruments and matched to each other based on the time data.

In general, the time data of the probe device and/or the preurethral placement device can be matched with the times and captured measured pressure values of the capsule, taking into account an inaccuracy interval regarding the times. This way, it is possible to account for the fact that a patient may not actuate the probe exactly at the moment of urinary urgency but perhaps only with a delay, for example a delay of several seconds. If the measured pressure values of the capsule and/or the established loss of urine is/are inside such a set inaccuracy interval around the time when the probe is actuated or, preferably, prior to the time of the actuation of the probe, these values can be considered as matching the probe actuations.

To achieve optimum accuracy of a bladder-pressure measurement system for any of the previously described embodiments, meaning also for those systems that do not include a probing device and preurethral placement device, it is advantageous for the measurement electronics to record the individual measured pressure values at time intervals that are as narrowly spaced as possible. On the other hand, this desire for a high-level of time resolution data is accompanied by a large quantity of data that must be stored inside the measurement capsule. For example, one possible embodiment can provide that the measured pressure values are captured once to ten times per second, preferably four times per second.

To limit these amounts of data, a preferred embodied form of any of the embodiments of the bladder-pressure measurement system can provide that the measurement electronics include a mean value memory where a mean value can be stored that is, in particular, a moving average from a number of past measured pressure values, and the measurement electronics are set up further to capture measured pressure values in time intervals, particularly periodically.

According to the invention, furthermore, the current measured pressure value is compared to a mean value in the mean value memory, whereupon, depending on the result of the comparison, the currently measured pressure value is then incorporated in the newly averaged mean value, and/or the value is stored in a separate memory or area of the memory, particularly for later retrieval.

For example, the measured pressure value can be incorporated only in the averaged mean value, but not permanently stored in a separate memory for later retrieval, when the current is measured pressure value shows only a deviation that is smaller than the preset mean value threshold. Only if the deviation is greater than the preset threshold value, can it be envisioned that the current measured pressure value is stored separately, because, as expected, it must be linked to detrusor instability, and wherefore this measured value must be, in particular, excluded from the averaging step.

Correspondingly, using such a bladder-pressure measurement system and/or the manner in which the measuring system is implemented, it is possible to considerably reduce the amount of needed memory space, because most of the measured values, namely those that are within a predetermined range of the mean value, are incorporated only for the further averaging of the mean value, otherwise they are discarded or not stored any further. Only remarkable values, meaning values that significantly deviate from the mean value and/or that exceed the threshold value, are stored separately for analysis at a later time, thereby reducing the required storage space in the measurement capsule.

In one embodiment, a memory is used only for storing a single measured pressure value, where the current measured pressure value is entered, and an earlier measured pressure value is possibly overwritten, if the current measured value exceeds a predetermined threshold value.

Another embodiment can provide that only those measured values are stored in a memory that exceed a preset threshold value. The memory comprises these measured values, preferably, in the order of their occurrence over time; and in a further improvement, these values are stored with additional time data from an internal clock.

One embodiment of the invention will be described in detail below based on one FIGURE.

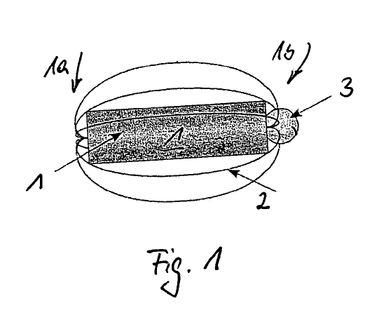

FIG. 1 shows a schematic view of a representation of a measurement capsule 1. This measurement capsule 1 has an essentially cylindrical housing with a preferably circular cross-section that is perpendicular relative to its longitudinal extension. Preferably, the diameter measured perpendicularly relative to the longitudinal extension of this measurement capsule between the ends 1a and 1b is smaller than 8 mm to allow for the placement of such a measurement capsule, for example, by standard cystoscopes.

As locking elements according to the invention, the presently shown embodiment has a plurality of spring wires 2 that extend between ends 1a and 1b of the measurement capsule in the longitudinal direction thereof. The extension therein is like magnetic field lines between the poles of a bar magnet. This means, in the currently described embodiment, preferably, the spring wires enter the areas at the ends 1a and 1b on the frontal side substantially perpendicularly. However, this is not necessarily the case in every embodiment.

Moreover, a further consequence is that, due to the spring-elastic property of these spring wires 2, they automatically achieve a maximum spacing from the surface of the measurement capsule in the central region between the ends 1a and 1b of the measurement capsule by the internal spring tension thereof.

The spring wires 2 that are, in the present embodiment, provided at evenly divided angles around the external cross-section of the measurement capsule can be placed against the external area of the measurement capsule 1 by pulling the measurement capsule 1 into the open end of a cystoscope, such that they enlarge the external cross-section of the measurement capsule 1 only insubstantially, essentially only by double the diameter of an individual wire.

To pull the measurement capsule 1 into the open end of a cystoscope or catheter, the measurement capsule 1 of the presently shown embodiment includes at the ends thereof, presently on end 1b, a gripping aid 3 that can be captured, for example, with tongs through the cystoscope. After the capturing step, the end 1b is therefore pulled into the hollow end of the cystoscope so that the internal wall of this hollow end region presses the wires 2 automatically against the capsule.

Therefore, it can be ascertained that a measurement capsule 1 according to the invention that is placed inside the bladder reaches an external cross-section that is large enough to prevent any inadvertent entry of the measurement capsule 1 into the urethra. Still, the external cross-section can be reduced in size such that the measurement capsule 1 according to the invention can be placed or recovered through the hollow end of the cystoscope.

In one embodiment not shown here it is possible to provide that the illustrated measurement capsule 1 is covered, at least locally, by an elastic sleeve that is, in turn braced from the inside by the spring wires.

Therefore, this sleeve raises itself automatically as soon as the measurement capsule of the type according to the invention has been expelled from the hollow end of the cystoscope. It is provided therein that the sleeve includes at least one opening, or at least it does not seal the measurement capsule, such that it is possible for a pressure equalization to occur between the inside and the outside of the sleeve that allows, on the one hand, for erecting the sleeve and, on the other hand, for the bladder pressure to be measured inside the measurement capsule, because the capsule is not shielded by the sleeve from the pressure that prevails inside the bladder.

Moreover, it must be generally provided that the pressure sensor that is provided inside the measurement capsule is able to feel the pressure outside of the measurement capsule.

For the measurement capsule according to the invention, it has proved especially advantageous to use for an external diameter that is smaller or equal 8 mm measurement electronics that comprise a controller of type MSP 430 F 2012 by Texas Instruments. The controller has the advantage of particularly low power consumption per measured value, such that this level of power consumption can be accommodated, for example, by a coin cell battery of type 319 having a diameter of only 5.8 mm.

Conceivable as a pressure measurement sensor is, for example, the sensor type BMP 085 by the company Bosch Sensortec. This product type is a barometric sensor that corresponds, however, to the pressure measuring range typically encountered in long-term urometry applications. In the alternative, there is the possibility of using a sensor of the Freescale MPL 115 A2 type.

Conceivable as a memory for the intermediate storage of the measured values, particularly together with the time values and/or a moving average value is, for example an EEPROM that has the advantage that it is nonvolatile even in the event of a power outage and therefore able to preserve the measured values. The measurement capsule described here can be used in connection with the presently not shown probe device for the purpose of implementing the method according to the invention.

The preceding concrete embodiment is not intended to limit the scope of the invention and serves merely as an example to describe the invention in more concrete terms, thereby outlining the functionality of the measurement capsule despite the small dimensions thereof.

1. A bladder-pressure measurement system comprising a measurement capsule that can be placed inside the bladder of a human being, the system comprising:
a pressure sensor;
measurement electronics that can capture and store measured pressure values provided by the pressure sensor;
a probe device having a probe that can be manually to actuated, the probe device and the measurement capsule including respective internal chronometers that are synchronized with each other or at least that can be synchronized with each other, the measurement capsule being configured such that it captures and stores measured pressure values together with time data from the chronometer thereof, the probe device being configured such that it captures and stores an actuation of the probe together with time data from the chronometer thereof.

2. The bladder-pressure measurement system according to claim 1, further comprising:
a preurethral placement device that can be placed in front of the outlet of the urethra of the patient and that comprises a sensor for detecting any loss of urine, wherein the preurethral placement device further includes an internal chronometer that is synchronized, or at least that can be synchronized, with at least one of the above-described chronometers, and in that the preurethral placement device is configured such that it captures any loss of urine together with time data from the chronometer thereof.

3. The bladder-pressure measurement system according to that claim 1, wherein the measurement electronics include a memory where a measured pressure value can be stored, the measurement electronics being configured such that the stored measured pressure value is overwritten by a newly detected measured pressure value, provided the new measured pressure value exceeds a preset threshold value, and the new measured pressure value is otherwise discarded.

4. The bladder-pressure measurement system according to claim 1, wherein the measurement electronics include a mean-value memory where a mean value, particularly a moving average, can be stored from a number of formerly measured pressure values, and the measurement electronics is configured such that a measured pressure value is captured in respective time internals, particularly periodically, compared to a mean value, and the currently measured pressure value is incorporated, depending on the results of the comparison, into the averaged value that is to be newly formed, or it is to be stored in a memory or area of the memory.

5. A method of measuring bladder pressure comprising the steps of:
capturing and storing measured pressure values in measurement electronics by a measurement capsule placed inside the bladder of a human being,
using a probe device that comprises a probe that can be manually actuated, capturing the actuations of the probe together with respective time data by a chronometer of the probe device that is synchronized with a chronometer of the measurement capsule.

6. The bladder-pressure measurement system comprising:
a measurement capsule that can be placed inside the bladder of a human being, and that includes a pressure sensor and a measurement electronics by means of which measured pressure values provided by the pressure sensor can be captured and stored, particularly according to claim 1, wherein at least one locking element that has force applied thereto, is provided on the measurement capsule that can be applied against the effective force against the measurement capsule and that can be automatically pushed away from the measurement capsule, at least locally, particularly by means of which the external cross-section of the measurement capsule can be at least locally enlarged.

7. The bladder-pressure measurement system according to claim 6, wherein the at least one locking element can be placed against the measurement capsule by at least one sleeve that surrounds the measurement capsule, and in that it can be pushed away after the removal of the sleeve from the measurement capsule.

8. The bladder-pressure measurement system according to claim 7, further comprising:
a cystoscope or an application aid whose frontal hollow end region accommodates/can accommodate the measurement capsule and constitutes the above-described sleeve.

9. The bladder-pressure measurement system according to claim 7, wherein a plurality of the locking elements is evenly distributed at least angularly around the measurement capsule.

10. The bladder-pressure measurement system according to claim 9, wherein the locking elements are configured as spring wires that each extend along a longitudinal extension of the measurement capsule from the one end thereof to the other end thereof, and that are provided in the central region of the measurement capsule as spaced thereto, due to the effect of the spring force.

11. The bladder-pressure measurement system according to claim 9, wherein the locking elements are each configured as a two-dimensional element that is fastened in an articulated manner by at least one end to the measurement capsule.

12. The bladder-pressure measurement system according to claim 1, wherein the locking elements are covered by an elastic cover.

13. The bladder-pressure measurement system according to claim 1, wherein a gripping aid is provided at least on one of the ends of the longitudinally extended measurement capsule, and the measurement capsule can be pulled into the sleeve by a gripping element.
